(19) United States
(12) Patent Application Publication
(10) Pub. No.: US 2017/0191101 A1
(43) Pub. Date: Jul. 6, 2017

(54) CLARIFICATION OF MAMMALIAN CELL CULTURE

(71) Applicant: Regeneron Pharmaceuticals, Inc., Tarrytown, NY (US)

(72) Inventors: Benjamin ADAMS, Pleasantville, NY (US); Andrew TUSTIAN, Tarrytown, NY (US); Hanne BAK, New York, NY (US)

(21) Appl. No.: 15/126,790

(22) PCT Filed: Mar. 17, 2015
(86) PCT No.: PCT/US15/20862

§ 371 Date: Sep. 16, 2016

Related U.S. Application Data
(60) Provisional application No. 61/954,456, filed on Mar. 17, 2014.

(51) Int. Cl. C12P 21/00; C12N 5/00

(52) U.S. Cl. CPC C12P 21/00; C12N 5/0081

(57) ABSTRACT

Disclosed is an improved method for clarifying a cell culture during the manufacture of a protein. The method includes the step of transiently reducing the pH of a cell culture, followed by a holding step for a period of time, followed by the neutralization of the cell culture prior to clarification by centrifugation.

### FIELD

The invention is generally directed to methods of producing biological molecules from cultured cells. Specifically, the invention is directed to an improved method of clarifying cell cultures for the production of biological molecules.

### BACKGROUND

The drive for high product titer mammalian cell culture of recombinant proteins, especially monoclonal antibodies, has increased cell density and process-related impurity levels thus placing a larger burden on traditional downstream clarification and purification operations. Controlled flocculation and precipitation of mammalian cell culture suspensions by acidification has been employed to both enhance clarification throughput and aid removal of process related impurities such as host cell protein and DNA (Lydersen et al., 1994, Annals of New York Academy of Sciences 745, 222-31).

Traditional acid precipitation typically necessitates bulk harvest pool neutralization before any capture chromatography can be performed, which can complicate production-scale downstream processing and hinder adoption of a more efficient continuous processing strategy (U.S. Pat. No. 7,855,280B2 and US Patent Application No. 20130012689A1). Thus, a significant unmet need exists for the efficient and cost-effective large scale production of proteins. An improved method of clarifying cell cultures for the large scale production of proteins is disclosed.

### SUMMARY

An improved method of clarifying a cell culture, wherein the cell culture is acidified, held, and neutralized prior to clarification, is disclosed. This improvement, termed transient pH treatment, enables continuous processing of protein production, higher productivity, and reduces process-intermediate tank requirements. A method of manufacturing a protein, which encompasses the transient pH treatment, is also disclosed. Also, a protein, which is manufactured according to a method that employs the transient pH treatment process, is also disclosed.

In one aspect, the invention provides a method of clarifying a cell culture comprising the steps of (a) lowering the pH of the cell culture, (b) holding the cell culture, (c) neutralizing the cell culture, and then (d) clarifying the cell culture.

In one embodiment, the pH of the cell culture is lowered to pH 4.3±0.2 at step (a). The pH may be lowered by adding phosphoric acid to the cell culture. The phosphoric acid may be in a concentrated form, such as 2M, prior to adding it to the cell culture.

In one embodiment the cell culture is held for 30 to 60 minutes at 15 to 20° C. after acidification and before neutralization.

In one embodiment, after the culture is held, the cell culture is neutralized by adding a buffer to the cell culture to bring the pH of the cell culture to a pH of 5.8 to 6.2 (6.0 ±0.2). The buffer may be tris(hydroxymethyl)aminomethane) (“tris”), which may be in a concentrated form, such as 2M, prior to adding it to the cell culture.

In another embodiment, after the cell culture is neutralized, the cell culture is clarified by centrifugation, which results in the formation of a pellet fraction, which contains cell debris and precipitated matter, and the formation of a clarified supernatant fraction, which contains soluble material comprising a substance of interest. The neutralized cell culture may be centrifuged at a force of at least 7,000 times the force of earth's gravity (i.e., >7,000 g), such as 7,390 g. The centrifugation may be performed in a disk stack centrifuge, such as a BTPX710 or a LAPX404 (Alfa Laval) model centrifuge (Alfa Laval Corporate AB, Lund, Sweden). The neutralized cell culture may be fed into the centrifuge at a rate of 2,000 liters per hour (L/h).

In another aspect, the invention provides a method of manufacturing a protein comprising the steps of (a) obtaining a cell culture, which comprises protein, cells, and media; (b) lowering the pH of the cell culture; (c) holding the cell culture; (d) neutralizing the cell culture; and then (e) forming a pellet fraction and a clarified supernatant fraction. The protein is generally found within the clarified supernatant fraction.

In one embodiment, the protein is produced by the cells and secreted into the culture medium. The cells may naturally produce the protein, or the cells may harbor a heterologous genetic element that encodes a recombinant protein and therefore produce a heterologous protein.

In one embodiment, the protein is an antigen-binding protein. An antigen binding protein may be any molecule that contains an amino acid polymer (i.e., a peptide, a polypeptide, a higher order complex of peptides or polypeptide chains, and the like), which can bind to an antigen. The antigen binding protein may be an antibody, such as a monoclonal antibody that preferentially binds to a particular epitope. The antigen binding protein may be an antibody that is known in the art as a bispecific antibody. A bispecific antibody may bind to two different epitopes. Those epitopes may be on the same polypeptide or protein, or may be on different polypeptides or proteins.

In one embodiment, the cells of the cell culture are mammalian cells. Those mammalian cells may be Chinese hamster ovary (CHO) cells, or a CHO cell derivative, such as CHO-K1 cells. The cells may harbor a genetic construct that encodes the protein, which may be heterologous or heterologous and recombinant.

In one embodiment, the clarified supernatant, which harbors the protein, may be subjected to steps designed to increase the relative purity of the protein. After clarification, the clarified supernatant may be subjected to any one or more of the steps of depth filtration, polish filtration, affinity chromatography (e.g., protein A chromatography), ion exchange chromatography (e.g., anion exchange, cation exchange, mixed bed, and the like), viral inactivation, concentration, diafiltration, and the like.

In another aspect, the invention provides a protein that is produced according to any of the methods described above (supra).

### DRAWINGS

FIG. 1 depicts a flow chart representing the different strategies for harvesting a monoclonal antibody. The disclosed transient treatment is compared to the traditional acidic precipitation and a no precipitation comparator method.

FIG. 2 depicts depth filter differential pressure and effluent turbidity profiles across depth filters for the three harvest strategies using monoclonal antibody B (mAb B).

FIG. 3 depicts normalized soluble aggregate for affinity eluate and viral inactivated pool as a function of three harvest strategies for monoclonal antibody A (mAb A) and B.

FIG. 4 depicts pool turbidity for both affinity eluate and viral inactivated pool as a function of three acid harvest strategies for mAb A and B.

FIG. 5 depicts distribution plots evaluating harvest pool host cell protein (HCP) and DNA log reduction during traditional acid precipitation (a) and transient pH treatment (b) for mAb B.

### DETAILED DESCRIPTION

### Methods

Depth filter load was produced via disk stack centrifugation, performed using an LAPX404 centrifuge (Alfa Laval; Alfa Laval Corporate AB, Lund, Sweden), scaled from an industrial centrifuge using sigma factor theory (Ambler, C M. 1961. Industrial and Engineering Chemistry 53, 430-45). All depth filters were loaded at a constant flux of 150 LMH. Along with depth filter pressure differential, turbidity of the depth filter effluent was monitored to assess particulate breakthrough using a 2020 wi turbidity meter (LaMotte). Depth filter capacity was defined as a 30 psi pressure differential on the filter or an effluent filtrate turbidity in excess of 50 Formazin Nephelometric Units (FNU). Host cell protein (HCP) and DNA were measured in harvest pools using an anti-CHO HCP ELISA kit (Cat. # F550, Cygnus Technologies) and qPCR respectively.

Samples from each strategy were captured using Protein A affinity chromatography to evaluate impurity levels in both the affinity pool and post-viral inactivation via low pH hold. All Protein A runs were conducted loading to 5% breakthrough +/−5%. Viral inactivated pool was adjusted to an intermediate, slightly acidic pH prior to filtration. For the affinity eluate pool and viral inactivated pool, turbidity was estimated using a Genesys 10UV spectrophotometer (Thermo Scientific) reading the optical density at 340 nm wavelength. Soluble aggregate was measured using an analytical SEC column attached to a UPLC.

Table 1 provides an overview of the strategies performed for each monoclonal antibody (mAb A and mAb B). Data shown in the FIGS. 2-5 represent the average value of the 500 L bioreactors with error bars representing the range of results from the various 500 L bioreactors. For soluble aggregate, viral inactivated pool was tested on only two bioreactors for the comparator and the transient strategy. The 40 L bioreactors were tested for host cell protein and DNA in the harvest pool without affinity capture.

The improved transient approach employed an acidification step, followed by a hold, followed by a neutralization step (see Table 2). Following neutralization, the harvest supernatant is subjected to depth filtration (see Table 3).

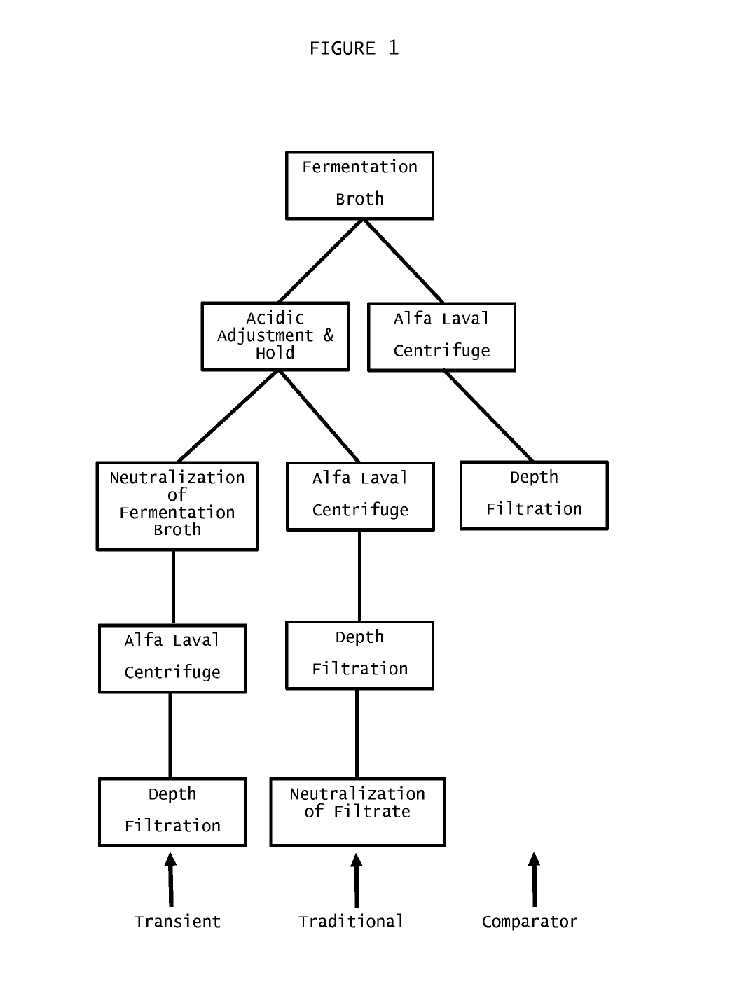

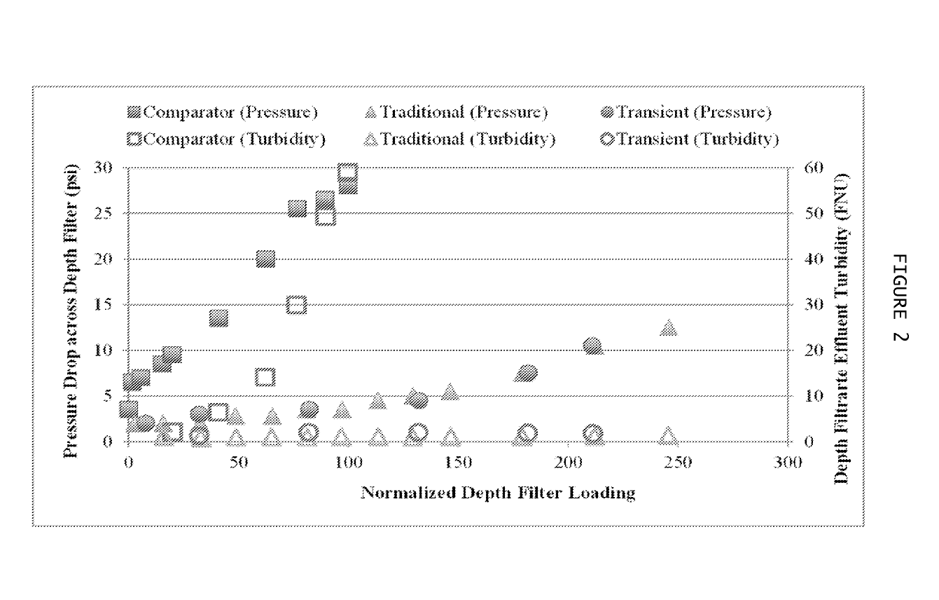

TABLE 1
Harvest strategies performed on bioreactors using two monoclonal
antibodies. See also FIG. 1.

| | |
|---|---|
| mAb A | mAb B |

| | | | |
|---|---|---|---|
| Scale (L) | 500 | 500 | 40 |
| Comparator | N = 1 | N = 3 | N = 13 |
| Traditional | | N = 1 | |
| Transient | | N = 3 | |

TABLE 2
Transient approach for acid precipitation

| Step | Description | Buffer Type | Volume |
|---|---|---|---|
| 1 | Record initial pool volume | N/A | N/A |
| 2 | Adjust to pH 4.1-4.4 | 2M Phosphoric Acid | 40 ± 5 g/kg |
| 3 | Hold for 30-40 min | N/A | N/A |
| 4 | Adjust to pH 5.8-6.2 | 2M Tris Base | 30 ± 5 g/kg |
| 5 | Measure PCV and titer | N/A | N/A |

TABLE 3
Recommended centrifugation parameters

| Parameter | Specification |
|---|---|
| Bowl Speed (rpm) | 5700 (7390 g) |
| Feed Flow Rate (L/h) | 2000 |
| Q/Σ | 1.24 × 10⁻⁸ (Q/cΣ = 3.1 × 10⁻⁸) |
| Feed Pressure (psi) | 5-20 |
| Back-Pressure (psi) | 15-30 |
| Discharge Interval (seconds) | 120 |

### Results

Both traditional acid precipitation and transient pH treatment resulted in comparable depth filter pressure profiles and capacities at 500 L pilot-scale. Both the traditional acid precipitated and transient pH treated material was processed using FOHC grade depth filters (EMD Millipore), designed for acid precipitated CHO culture (EMD Millipore Literature PF1119EN00 (2013)). The comparator material was processed over Regeneron's platform depth filter for non-acid precipitated material. A normalized capacity in excess of 200 was observed for both pre-treatments options, with the comparator material capacity at 100. See FIG. 2.

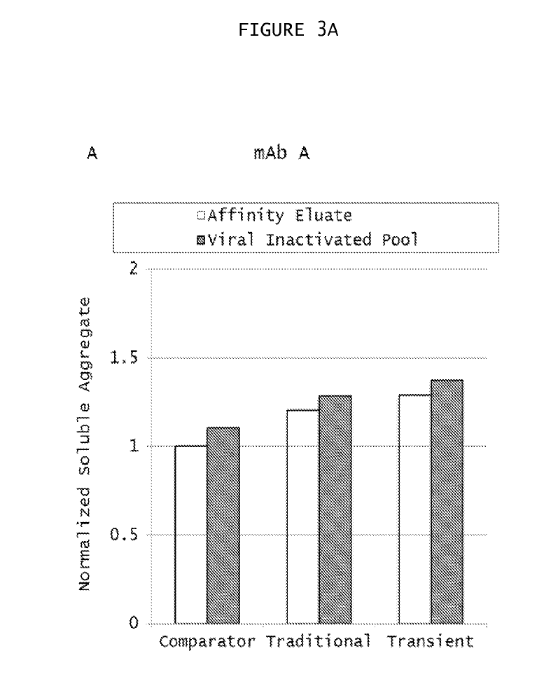

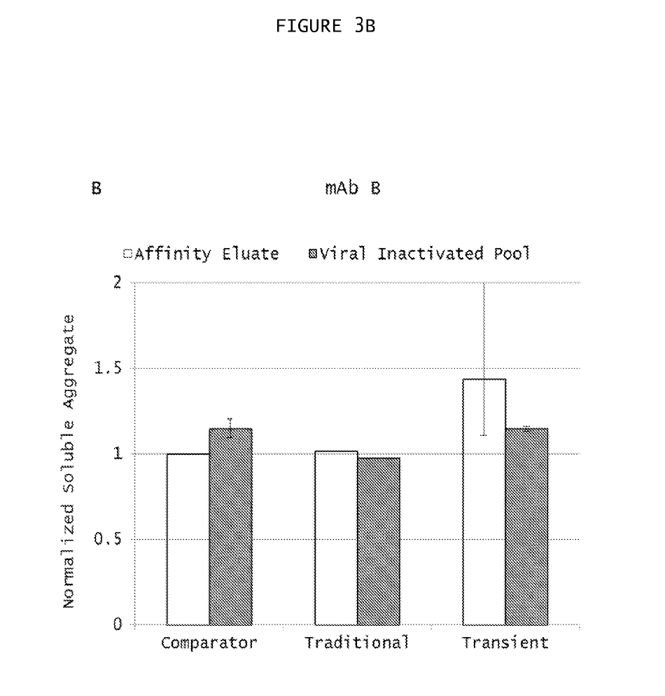

For mAb A, a modest increase in soluble aggregate in affinity eluate and viral inactivated pool is noted with both acid precipitation strategies. In contrast to this, for mAb B aggregate levels in viral inactivated pools were comparable for all three harvest strategies. These data suggests that while susceptibility to aggregate during acid precipitation is mAb dependent, the transient strategy does not generate more aggregate than traditional acid precipitation. See FIG. 3.

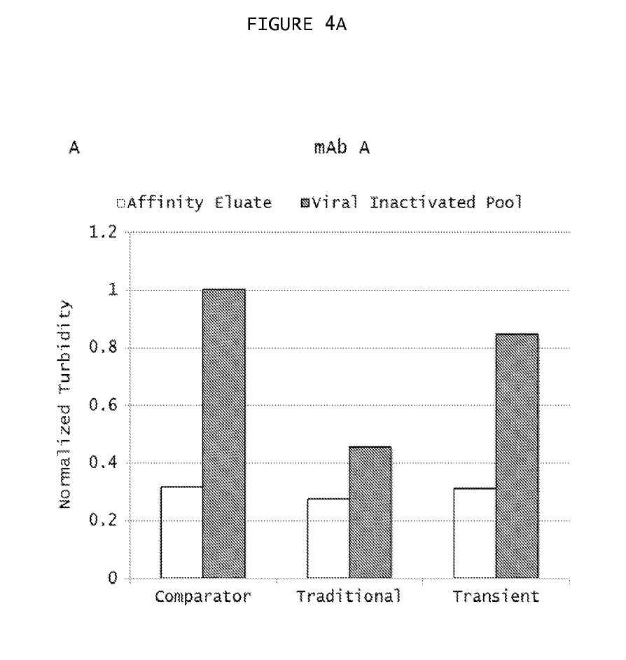

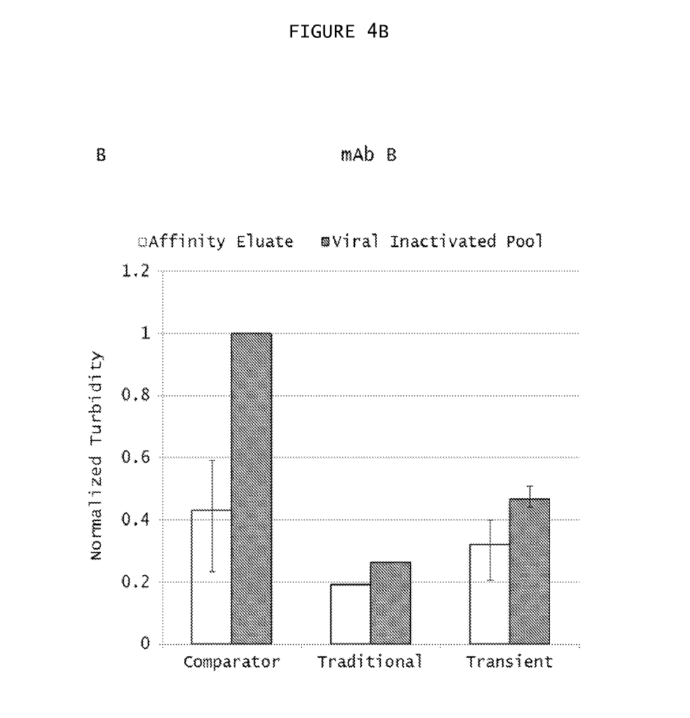

Eluate and viral inactivated pool turbidity is decreased by both acid precipitation strategies, suggesting improved process-related impurity removal as compared to the comparator feed stream (see Shukla et al., 2005. Bioprocess International 3(5), 36-45; and Yigzaw et al., 2006. Biotechnology Progress 22, 288-96). This effect is more pronounced with the traditional strategy. Specifically, for mAb A, traditional acid precipitation and transient pH treatment strategies resulted in viral inactivated pool turbidity 45% and 85% that of the comparator strategy, respectively. For mAb B, traditional acid precipitation and transient pH treatment strategies resulted in viral inactivated pool turbidity 26% and 47% that of the comparator strategy, respectively. See FIG. 4.

HCP and DNA levels were measured in cell culture and depth filtrate to isolate removal based solely on the two acid precipitation strategies. pH adjustments were performed in 40 L bioreactors (N=13) and samples of each strategy were spun down in a bench top centrifuge scaled using the sigma factor theory (see Singh et al., 2013. Biotechnology and Bioengineering. 110, 1964-72.). Disk stack centrifugal shear was not mimicked in this study as a micro-scale shear device was not available (see Boychyn et al., 2004. Bioprocess and Biosystems Engineering 26, 385-91). HCP levels were comparable between both acid precipitation strategies, resulting in an average 0.1 log reduction factor (LRF). A traditional acid precipitation strategy resulted in a 1.3 log increase in DNA clearance as compared to the comparator and transient strategy. See FIG. 5.

### Conclusion

A comprehensive evaluation of a modification to traditional acid precipitation in which cell culture is acidified, held, and neutralized prior to clarification was performed with the following outcomes.

Filter throughput for the transient strategy more than two-fold increased as compared to the comparator strategy when clarified post centrifugation. Furthermore, filter throughput for the transient strategy was comparable to a traditional acid precipitation strategy when clarified post centrifugation.

Equivalent aggregate levels in virally inactivated Protein A pools were observed for both acid precipitation strategies. Susceptibility to aggregation during acid precipitation was monoclonal antibody dependent as mAb A acid precipitation resulted in 30-40% aggregate increase over comparator, whereas no increase was observed for mAb B.

Up to a 60% reduction in viral inactivated pool turbidity was observed from the transient acid precipitation strategy as compared to the comparator strategy while, the transient strategy resulted in a two-fold increase in viral inactivated pool turbidity compared to traditional acid precipitation for mAb A and B.

Overall, a pilot-scale study using two monoclonal antibodies has indicated the transient acid precipitation strategy is comparable to traditional acid precipitation in that it increases depth filter capacities, decreases affinity pool turbidity, and provides some DNA removal as compared to non-acid precipitated harvest. The transient strategy facilitates downstream processing compared to traditional acid precipitation by removing the necessity for neutralization of the harvest bulk pool prior to affinity capture. Therefore the strategy allows capture to commence before harvest is finished, facilitating continuous processing and process intensification.

What is claimed is:

1. A method of clarifying a cell culture comprising: (a) lowering the pH of the cell culture; (b) holding the cell culture at the lower pH; (c) increasing the pH of the cell culture; and then (d) clarifying the cell culture.

2. The method of claim 1, wherein the pH of the cell culture is lowered to pH 4.3±0.2 at step (a).

3. The method of claim 2, wherein the pH of the cell culture is lowered at step (a) by adding phosphoric acid to the cell culture at stop (a).

4. The method of claim 3, wherein the pH of the cell culture is lowered by adding 2M phosphoric acid to the cell culture at step (a).

5. The method of claim 1, wherein the cell culture is held at step (b) for 30 to 60 minutes at 15 to 20° C.

6. The method of claim 1, wherein the pH of the cell culture is increased at step (c) by adding tris(hydroxymethyl)aminomethane) (tris) to the cell culture to attain a pH of 6.0±0.2.

7. The method of claim 6, wherein the pH of the cell culture is increased at step (c) by adding 2M tris to the cell culture.

8. The method of claim 1, wherein the cell culture is clarified at step (d) by disk stack centrifugation, wherein a pellet fraction and a clarified supernatant fraction are formed.

9. The method of claim 8, wherein the cell culture is clarified by centrifugation at ≧7,000 g and at a feed flow rate of 2,000±500 L/h.

10-11. (canceled)

12. A method of manufacturing a protein comprising: (a) obtaining a cell culture, which comprises protein, cells, and media; (b) lowering the pH of the cell culture; (c) holding the cell culture at the lower pH; (d) increasing the pH of the cell culture; and then (e) forming a pellet fraction and a clarified supernatant fraction.

13. The method of claim 12, wherein the cells secrete the protein into the media.

14. The method of claim 12, wherein the protein is an antigen-binding protein.

15. The method of claim 14, wherein the antigen-binding protein is an antibody.

16. The method of claim 15, wherein the antibody is capable of binding binds more than one epitope.

17. The method of claim 16, wherein the antibody is binds to an epitope on one antigen and an epitope on another antigen.

18. The method of claim 12, wherein the cells are mammalian cells.

19. The method of claim 18, wherein the cells are Chinese hamster ovary (CHO) cells.

20. The method claim 12 further comprising subjecting the clarified supernatant fraction after step (e) to depth filtration.

21. (canceled)

22. A protein produced according to the method of claim 12.

23. The protein of claim 22, wherein said protein is an antigen-binding protein.
